(19) United States
(12) Patent Application Publication
(10) Pub. No.: US 2023/0122876 A1
(43) Pub. Date: Apr. 20, 2023

(54) Chemical Reagents for the Activation of Polysaccharides in the Preparation of Conjugate Vaccines

(71) Applicant: Fina BioSolutions, LLC, Rockville, MD (US)

(72) Inventor: Andrew Lees, Silver Spring, MD (US)

(73) Assignee: Fina BioSolutions, LLC, Rockville, MD (US)

(21) Appl. No.: 17/963,436

(22) Filed: Oct. 11, 2022

Related U.S. Application Data
(60) Provisional application No. 63/257,749, filed on Oct. 20, 2021.

(51) Int. Cl. A61K 31/715; A61K 31/25; C07K 14/195

(52) U.S. Cl. CPC A61K 31/715; A61K 31/25; C07K 14/195

(57) ABSTRACT

This invention provides novel reagents for cyanating polysaccharides in aqueous or part aqueous solutions so that they may be covalently linked to peptides or proteins either directly or through a spacer. These reagents include 1-cyano-4-dimethylaminopyridinium bromide, or a functional derivative or modification thereof. The examples illustrate the use of these reagents with a variety of polysaccharides and proteins showing that the methods are generally applicable.

### REFERENCE TO RELATED APPLICATIONS

This application claims priority to U.S. Provisional Application No. 63/257,749 filed Oct. 20, 2021, the entirety of which is incorporated by reference.

### BACKGROUND

### Field of Invention

This invention is directed to reagents and methods of conjugating proteins to carbohydrates and, in particular, the processes, chemicals, and compositions used in the manufacture of immunological compositions and vaccines, and also the immunological compositions and vaccines manufactured by the process.

### Description of the Background

Vaccines that contain protein covalently linked to carbohydrate have proven remarkably successful in inducing an immune response to the carbohydrate moiety. Examples of such vaccines, known as “conjugates” are available for Haemophilus influenzae type b (e.g., ActHib, Hiberix), Neisseria meningiditis types A C W and Y (e.g., Menactra) and S. pneumoniae (e.g., PREVNAR®, SYNFLORIX®). For the protein to be linked to the carbohydrate, the latter generally needs to be activated so that it can be reacted with the protein, either directly or via a spacer (Dick, W.E. Jr and Beurret, M. Glyconjugates of bacterial carbohydrate antigens. A survey and consideration of design and preparation factors. In: Conjugate Vaccines (Eds Cruse, J.M. and Lewis, R.E.). Karger, Basel, 1989. One means of activation is through oxidation of the carbohydrate to produce aldehydes, which are then linked to lysines on protein through reductive amination. In other cases, the protein is first functionalized with hydrazides or aminooxy groups, which are subsequently reacted with aldehydes on the carbohydrate (Lees, A. Use of amino-oxy functional groups in the preparation of protein-polysaccharide (PS) conjugate vaccines U.S. Pat. Publication No. 2005/0169941; which is incorporated by reference). Another method for activating polysaccharides is with the use of cyanogen bromide, to form a cyano-ester on the polysaccharide which is subsequently reacted with a spacer molecule such as adipic dihydrazide. The functionalized polysaccharide is then reacted with the protein. Improved methods for cyanylating polysaccharides use 1-cyano-4-dimethylaminopyridine tetrafluoroborate (CDAP—TFB also referred to herein as CDAP) (see U.S. Pat. Nos. 5,651,971; 5,693,326; and 5,849,301, which are each incorporated by reference). The chemical structure of CDAP is depicted in FIG. 1 wherein the counter ion is tetrafluoroborate. During cyanylation, CDAP links to a PS forming an activated intermediate molecule, which then reacts with a peptide and forming a conjugate PS-peptide molecule. Thus, the protein is linked directly to the polysaccharide via the cyanylation reaction (see FIG. 2). Intermediates in the cyanylation reaction include pyridinium isourea as well as formation of a cyanate ester. CDAP can also be used to functionalize the polysaccharide with a spacer, which is subsequently linked to the protein. Hydrazide or aminooxy functionalized proteins can also be linked to CDAP activated polysaccharides (see e.g., U.S. Pat. No. 5,849,301, which is incorporated by reference).

The use of CDAP is disclosed in WIPO Publication No. WO99/036777 (corresponding to U.S. Pat. No. 6,916,666 which issued Jul. 12, 2005, and is incorporated by reference) for coupling polystyrene particles, and also in WIPO Publication No. WO2002/23774 (corresponding to U.S. Pat. No. 7,102,024, which issued Sep. 05, 2006, and is incorporated by reference) for coupling oligosaccharides. Both of these references refer to CDAP—TFB, as the CDAP compound is referenced as soluble in acetone or referenced to publications that disclose only CDAP—TFB (e.g., see Lees et al., Vaccine 14:190 (1996) and Lees et al., Vaccine 18:1237, 2000).

Although CDAP represents a great stride in the development of vaccines, CDAP is difficult to work with. Stock solutions of the compound at high concentrations, typically 25-150 mg/ml are prepared, and added to the polysaccharide solution. However, CDAP—TFB is poorly soluble in aqueous solutions and the reagent needs to be solubilized in organic solvents such as acetonitrile or acetone, and thus, toxic to work with and requiring special disposal. Also, as most antigenic compounds require aqueous chemical conditions, the fact that CDAP requires organic solvents and most antigens require aqueous conditions, this presents challenges in chemistry. Thus, there is an unmet need for an aqueous cyanylating compound that is useful in the manufacture of immunological agents, diagnostics, vaccines, and similar chemical compounds.

### SUMMARY OF THE INVENTION

The present invention overcomes the problems and disadvantages associated with current strategies and designs in the manufacture of immunological compositions and diagnostic agent and provides new tools and methods of protein conjugation especially for vaccine manufacture.

One embodiment of the invention is directed to a 1-cyano-4-dimethylaminopyridinium bromide (CDAP—Br), or a functional derivative or modification, thereof. CDAP—Br maybe in the form of a dry power, or a liquid. Preferably CDAP—Br is reacted with a PS or similar chemical compound. The PS is activated by CADP—Br forming an activated PS, the PS—CDAP in a solution containing bromide ions. As both CDAP—Br and the antigen are in aqueous solutions, the activated compound is also in an aqueous solution.

Another embodiment of the invention is directed to a process of conjugation of a carbohydrate under aqueous conditions comprising: mixing a 1-cyano-4-dimethylaminopyridinium bromide (CDAP—Br), or a functional derivative or modification, thereof with the chemical compound to create an activated chemical compound; and mixing the activated compound with a second compound to create a conjugate. Preferably the conjugation process is performed in an aqueous solution wherein the CDAP compound is maintained in a bromide solution, preferably with no tetrafluoroboride (TFB). Preferably the PS is a natural or synthetic carbohydrate, polysaccharide, oligosaccharide, dextran, or combination thereof. Also preferably, the first chemical compound is a peptide, a polypeptide or a protein, which may be an antigenic or diagnostic molecule. The PS or activated PS may contain a second chemical compound such as, for example but not limited to a spacer or linker molecule such as adipic dihydrazide or hexane diamine. Conjugation may be direct or indirect with the additional of functional groups that facilitate conjugation. Functional groups may include amines, hydrazides and aminooxy groups, among others. The activated PS may also be linked to small molecules such as biotin, click reagents or a fluorescent molecule. Examples of click reagents are described in Bernhard Stump Chembiochem, 23 (16):e202200016, 2022 (incorporated by reference). The process preferably further comprises removing components with a lower molecular weight than the conjugate for example, by dialysis filtration, chromatography, or a combination thereof. The resulting conjugate is preferably a vaccine or a diagnostic agent. The process may further comprise including a linking compound between the activated compound and the second compound. Preferably the steps of the process, as well as the inclusion of a linker, are performed together, but each may be performed independently.

Another embodiment of the invention is directed to a process of conjugation in an aqueous composition comprising: reacting 1-cyano-4-dimethylaminopyridinium bromide with a polysaccharide to form an activated polysaccharide; reacting the activated polysaccharide with a first chemical compound; and forming a conjugate. Preferably the polysaccharide comprises a natural or synthetic carbohydrate, oligosaccharide, or a dextran, or a combination thereof, wherein the polysaccharide contains an epitope of a pathogen. Preferred pathogens comprises Staphylococcus spp., Methicillin-resistant Staphylococcus aureus, Haemophilus spp., Haemophilus influenzae type B, Klebsiella spp., Pseudomonas spp, Escherichia spp., Escherichia coli, Neisseria spp., Neisseria meningitides, Streptococcus spp., Streptococcus pneumococcus, Group B Streptococcus, Mycobacterium spp., Mycobacterium tuberculosis, Acinetobacter spp., Acinetobacter baumannii, Clostridium spp., Clostridium difficile, Burkholderia spp., Burkholderia cepacia, Vibrio spp., Salmonella spp., or Shigella spp. Preferably the polysaccharide or the activated polysaccharide contains a second chemical compound which may comprise a linker or spacer molecule. Preferred linker molecule comprises a homobifunctional or a heterobifunctional linker molecule. Preferably the first chemical compound comprises a natural or recombinantly or synthetically produced peptide, polypeptide, or protein, such as, for example, a natural or recombinantly produced diphtheria toxoid, diphtheria cross reactive material (CRM), CRM₁₉₇. tetanus toxoid, tetanus toxin heavy chain protein, Pseudomonas exoprotein A, Pseudomonas aeruginosa toxoid, Bordetella pertussis toxoid, or Clostridium perfringens toxoid. Preferably the 1-cyano-4-dimethylaminopyridinium bromide contains an approximate ratio of about one mole of 1-cyano-4-dimethylaminopyridinium per mole of bromide anion, and the process does not include tetrafluoroborate or organic solvents. Preferably, the process further involves removing components with a lower molecular weight than the conjugate by dialysis, filtration, chromatography, or a combination thereof.

Another embodiment of the invention is directed to a process of conjugation in an aqueous composition comprising: reacting 1-cyano-4-dimethylaminopyridinium bromide with a polysaccharide; reacting the activated polysaccharide with a biotin hydrazide; and reacting the activated polysaccharide with a first chemical compound forming a biotinylated polysaccharide conjugate. Preferably, the polysaccharide comprises a natural or synthetic carbohydrate, oligosaccharide, or dextran, or a combination thereof, and the first chemical compound comprises a natural or recombinantly or synthetically produced peptide, polypeptide, or protein such as, for example, a natural or recombinantly produced diphtheria toxoid, diphtheria cross reactive material (CRM), CRM₁₉₇. tetanus toxoid, tetanus toxin heavy chain protein, Pseudomonas exoprotein A, Pseudomonas aeruginosa toxoid, Bordetella pertussis toxoid, or Clostridium perfringens toxoid. Preferably the biotinylated polysaccharide conjugate is coupled to an affinity compound such as, for example but not limited to, an avidin, an avidin-like protein, a rhizavidin, a streptavidin, or a functional portion thereof. Preferably, the process further comprising removing components with a lower molecular weight than the conjugate by dialysis, filtration, chromatography, or a combination thereof.

Another embodiment of the invention is directed to a process of conjugation under aqueous conditions comprising: reacting 1-cyano-4-dimethylaminopyridinium bromide with a polysaccharide to form a cyanalyated polysaccharide; reacting the cyanalyated polysaccharide with a dibenzocyclootyne to a form a dibenzocyclootyne coupled polysaccharide; and reacting the dibenzocyclootyne coupled polysaccharide with a first chemical compound protein to form a conjugate. Preferably, the polysaccharide comprises a natural or synthetic carbohydrate, oligosaccharide, or a dextran, or a combination thereof. Also preferably, the dibenzocyclootyne coupled cyanalyated polysaccharide further comprises polyethylene glycol (PEG). Preferably, the first chemical compound comprises a natural or recombinantly produced diphtheria toxoid, diphtheria cross reactive material (CRM), CRM₁₉₇. tetanus toxoid, tetanus toxin heavy chain protein, Pseudomonas exoprotein A, Pseudomonas aeruginosa toxoid, Bordetella pertussis toxoid, or Clostridium perfringens toxoid.

Another embodiment of the invention is directed to an antigenic compound, an immunological compound, a diagnostic agent, or a vaccine produced by the processes disclosed herein. Preferably the immunological composition is a vaccine, such as, for example, a vaccine to treat or prevent a pathogenic disease or infection. Preferably the vaccine further comprises a pharmaceutically acceptable carrier which may include, but is not limited to water, saline, alcohol, saccharides, polysaccharides, oil, or combinations thereof, and/or an adjuvant which may contain or exclude aluminum chemical compounds.

Another embodiment of the invention is directed to a composition comprising a polysaccharide activated with 1-cyano-4-dimethylaminopyridinium in the presence of bromide anions. Preferably the bromide anions are present in an approximate 1:1 molar ration with molecules of CDAP.

Other embodiments and advantages of the invention are set forth in part in the description, which follows, and in part, may be obvious from this description, or may be learned from the practice of the invention.

### DESCRIPTION OF THE DRAWINGS

FIG. 1. CDAP—TFB.

FIG. 2. Depiction of chemical reaction in the activation of a polysaccharide (PS) by CDAP—TFB.

FIG. 3. CDAP—Br.

FIG. 4. Comparison of CDAP—TFB and CDAP—Br hydrolysis at pH 8 and 9.

FIG. 5. SEC HPLC monitoring of TNP—BSA/CDAP/Dextran showing the molecular weight (MW) of conjugates.

FIG. 6. ECOCRM®-Hib PRP conjugation analyzed by SEC MALS. Comparison of activation with CDAP—TFB vs. CDAP—Br.

### DESCRIPTION OF THE INVENTION

The introduction of conjugate vaccines to prevent infection caused by, for example Haemophilus influenzae type b (Hib), Streptococcus pneumonia, and Neisseria meningitides has saved hundreds of thousands of lives. These vaccines comprise a polysaccharide antigen of the infectious agent covalently linked to a protein, a process that converts the carbohydrate polymer from a T-cell independent to a T-cell dependent antigen. Preferably, the conjugation chemistry minimally disrupts critical antigenic elements of the polysaccharide. Cyanylation has been used to activate polysaccharide hydroxyls, first with cyanogen bromide and, more recently, with 1-cyano-4-dimethylamino pyridinium tetrafluoroborate (CDAP—TFB). CDAP—TFB has several advantages over CNBr; among them are reactivity at lower pH and the ability of CDAP—TFB-activated polysaccharides to directly bind to proteins. In the CDAP activation process, CDAP reagent is added to the polysaccharide solution, the pH is raised and maintained at about pH 9 for a few minutes, and either linker or protein is added. The reaction is usually complete in a few hours. CDAP is commonly prepared in an organic solvent, typically acetonitrile (methyl cyanide), but at large scale, in a biological GMP manufacturing suite, the use of this flammable, toxic solvent is problematic. Disposal of the CDAP compound in organic solvent is also an issue.

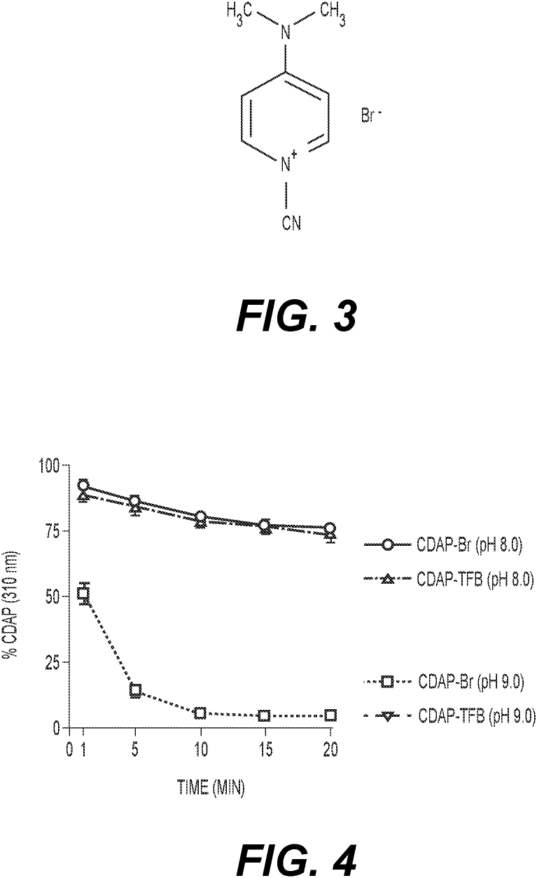

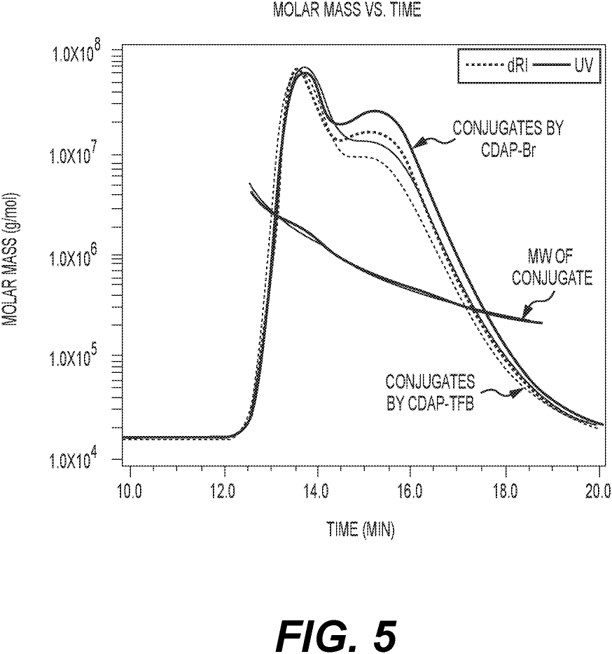

This disclosure is directed to a surprisingly discovered improvement to the original CDAP activation protocol with a CDAP analog, CDAP- bromide (1-cyano-4-dimethylaminopyridinium with bromide as the counter ion or CDAP—Br; see FIG. 3). CDAP—Br is soluble in aqueous acidic solutions (i.e., does not require organic solvents) to over 500 mg/ml and is stable in this solution. Activation of polysaccharides is performed in a manner comparable to CDAP—TFB, but the process is performed without organic solvent, which represents a major advantage at least in that now both the cyanylating agent and the agent are aqueous and activation can be performed under aqueous conditions. Thus, activation with CDAP—Br eliminates the need for organic solvents and the complications of toxicity and disposal of organic solvents. The substitution of CDAP—Br requires only minor modifications to the activation protocol to achieve nearly or virtually identical conjugation results allowing for this water-soluble reagent to be easily substituted for CDAP—TFB. Unlike CDAP—TFB, CDAP—Br is highly soluble in an aqueous medium, does not require a special chemical facility, and is safe to use as it does not involve a toxic flammable organic solvent, and has no disposal issues associated with the organic solvent as there is no organic solvent.

One disclosure of the invention is directed to a composition comprising CDAP—Br. The composition may be dry powder, or dissolved in an aqueous substance such as, for example, an aqueous salt and/or buffer solution. Preferably the composition comprises the activated PS in a bromide solution at room and reduced temperatures. The structure of the activated PS is basically the same as depicted in FIG. 2, except that the counterion is bromide and not tetrafluoroborate. Bromide anions are present in an approximate 1:1 molar ration with molecules of CDAP. One of the intermediates of the reaction is pyridinium isourea.

Another disclosure of the invention is directed to a process of conjugation that involves use of CDAP—Br to create an activated chemical compound. The activated compound is mixed with a second compound to create a conjugate. These steps may be performed independently or together and may include another compound as a linker between the two. Preferably the activated compound is mixed with a linker molecule which is subsequently reacted with a second compound to create a conjugate. Preferred linkers include but are not limited to hexanediamine, ethylenediamine, hydrazine, adipic dihydrazide, or 1,6-diaminooxyhexane.

Conjugation may be direct or indirect, meaning with or without the addition of functional groups that facilitate conjugation. Preferably, the chemical compound is a natural or synthetic carbohydrate, polysaccharide, oligosaccharide, or combination thereof. Preferably the second compound is a peptide, a polypeptide or a protein, and more preferably the second compound is an antigenic molecule for the preparation of a immunological agent, a vaccine, or a diagnostic reagent. The second chemical compound can be a small molecule such as biotin, click reagents, a labeling compound such as, for example but not limited to, a fluorescent molecule.

This disclosure provides novel reagents for cyanylating polysaccharides in aqueous or part aqueous solutions so that they may be covalently linked to proteins either directly or through a spacer. The examples illustrate the use of these reagents with a variety of polysaccharides and proteins showing that the methods are generally applicable.

The terms carbohydrate, polysaccharide, and oligosaccharide are used interchangeably in this application. Polysaccharides of the disclosure further include dextran molecules. The method can employ either natural or synthetic forms. Protein can refer to natural, recombinant or synthetic material including peptides. Other molecules besides protein can be used as the first chemical compound to link either directly or indirectly to the activated carbohydrate. Preferred peptides, oligopeptides, and proteins include, but is not limited to a natural or recombinantly produced T cell activating agents, diphtheria toxoid, diphtheria cross reactive material (CRM), CRM₁₉₇. tetanus toxoid, tetanus toxin heavy chain protein, Pseudomonas exoprotein A, Pseudomonas aeruginosa toxoid, Bordetella pertussis toxoid, or Clostridium perfringens toxoid.

Direct conjugation refers to linking of the protein to the activated carbohydrate without introducing additional functional groups. Indirect conjugation refers to the addition of functional groups which are used to facilitate conjugation. For example, carbohydrate can be functionalized with amines which are subsequently derivatized with bromoacetyl groups. The bromoacetylated carbohydrate is then reacted with thiolated protein. (Hermanson, GT, Bioconjugate Techniques, Academic Press, 2^{nd} ed, 2008). The term functionalization generally means to chemically attach a group to add functionality, for example, to facilitate conjugation. Examples include functionalization of proteins with hydrazides or aminooxy groups and functionalization of carbohydrate with amino groups.

Conjugation of the protein to the carbohydrate increases its molecular weight, which can be monitored using analytical size exclusion chromatography (SEC HPLC). The earlier the material elutes the higher the molecular weight, and protein is generally monitored by its absorbance at 280 nm. Thus, the shift of absorbance to an earlier elution time is an indication of an increase in molecular weight of the protein component and hence of conjugation. A TSK G4000 SEC column (Tosoh) or similar column was used for SEC HPLC in a Waters Alliance system equipped with Empower software. SEC MALS was performed using an Agilent 1100 HPLC with an Agilent UV detector, a TrEX refractive index detector (Wyatt Technologies) and a Dawn eight angle light scattering detector (Wyatt Technologies). Data collection and analysis was performed using Astra7 software (Wyatt Technologies).

Hydrazides were assayed using TNBS as generally described in: “Spectrophotometric determination of hydrazine, hydrazides, and their mixtures with trinitrobenzenesulfonic acid” Qi XY, Keyhani NO, Lee YC. Anal Biochem. 1988 Nov 15;175(1):139-44 and Vidal and Franci, J Immun. Meth 86:155, 1986 (both incorporated by reference).

Protein concentration was determined from its extinction coefficient and absorbance at 280 nm. Carbohydrate was assayed by the method of Monsigny M. et al. Anal Biochem 175:525-530, 1988 (incorporated by reference).

CRM₁₉₇ was produced in E. coli as described in U.S. Pat. Nos. 10,093,704, 10,597,664, and 11,060,123 (which are hereby entirely incorporated by reference) and referred to by the commercial product name ECOCRM®. BSA was purchased from SigmaAldrich (St. Louis, MO). Various polysaccharides were kindly provided by The Serum Institute of India (Pune, India) or Inventprise, LLC (Redmond, WA). CDAP—TFB was purchased from Sigma-Aldrich. CDAP—Br was a gift of Anders Holmberg (Sweden). Chemicals and reagents were of ACS grade or better.

The following examples illustrate embodiments of the invention but should not be viewed as limiting the scope of the invention.

### Example 1 Comparison of CDAP-TFB and CDAP-Br

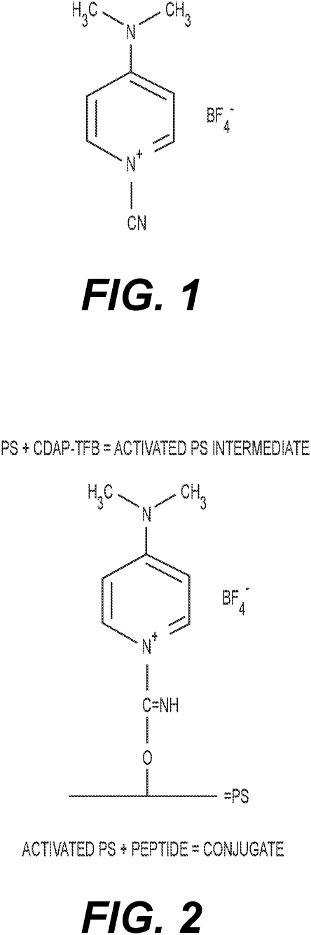

CDAP—TFB (chemical structure depicted in FIG. 1) is a white, crystalline substance, soluble in acetonitrile to greater than 100 mg/ml, but with slow dissolution and barely soluble at 50 mg/ml in 0.1 M HCl. CDAP—Br is formed by combining DMAP (dimethylaminopyridine) with cyanogen bromide resulting in a 1:1 molar ratio. Furthermore, when a CDAP—TFB solution in 0.1 M HCl was chilled on ice, it began to precipitate. In contrast CDAP—Br (chemical structure depicted in FIG. 3) is a white to pale-yellow powder. CDAP is prepared in a 1:1 molar ratio with bromide to form CDAP—Br. The bromide salt is poorly soluble at 50 mg/ml in acetonitrile but can be prepared at over 500 mg/ml in 0.1 M HCl at room temperature. Both forms of CDAP are stable in 0.1 M HCl for several hours.

### Example 2 Similar Rates of Hydrolysis

The rates of hydrolysis of CDAP—TFB and CDAP—Br at pH 8 and pH 9 were compared (FIG. 4). A stock solution of CDAP—TFB and CDAP—Br was prepared at 50 mg/ml in acetonitrile and 100 mg/ml in 0.1 N HCl, respectively. Each solution was kept on ice. For the stability study, 5 µL 0.1 N NaOH solution and then 5 µL CDAP—Br stock solution were added to 990 µL 0.1 M borate buffer (pH 9.0) or 0.1 M HEPES buffer (pH 8.0) in ice/water bath. At 1, 5, 10, 15 and 20 min after the addition, aliquots of reaction solution (10 µL) were taken out and quenched with 990 µL 0.1 HCl and stored at room temperature. The UV absorbance spectra of aliquoted samples from 250-400 nm were obtained with 0.1 N HCl as the blank. The UV absorbance at 310 nm of each sample was used for analysis of unhydrolyzed CDAP in the buffered solutions. Hydrolysis was followed by monitoring absorbance at 310 nm, a wavelength where CDAP absorbs but where DMAP, the hydrolysis product, has only a fraction of the absorbance. At the end of the reaction, the pH values of reaction solutions at 8.0 or 9.0 were confirmed as unchanged by a pH meter. All the experiments were independently performed in triplicate. Results are depicted in FIG. 4. The rates of hydrolysis over 20 minutes were indistinguishable for the two compounds. The similar hydrolysis rates for CDAP—TFB and CDAP—Br are an indication of similar stability in aqueous solutions. It is also an indication that there may be no need to adjust the amount of reagent in converting a process from CDAP—TFB to CDAP—Br.

### Example 3 Activation of Dextran

Next, the ability of CDAP—TFB and CDAP—Br to activate dextran was compared. Dextran is a model polysaccharide that is rich in hydroxyl groups. Dextran was activated with CDAP—Br or CDAP—TFB and reacted with adipic dihydrazide (ADH). The activation of dextran was evaluated by measuring the level of ADH derivatization. The activation protocol was as follows:

CDAP-Br was easily solubilized at 50 mg/ml in 50 mM HCl. CDAP—TFB was also solubilized at 50 mg/ml in 50 mM HCl, with extensive vortexing. Both reagent solutions were kept at room temp. Three ml of 10 mg/ml T2000 dextran was prepared in a 20 ml beaker with a micro stir bar, on ice. A pH probe in the dextran, with a separate temperature probe kept on ice, was used to monitor the pH. An auto-titrator, set to dispense 10 µl aliquots of 0.1 M NaOH, was positioned so that drops would land on the stir bar. At t= 0, 300 µl of the 50 mg/ml CDAP—TFB or CDAP—Br was added and NaOH was dispensed first to raise pH and then to maintain it at pH 9 +/- 0.1 for 15 min. At 15.5 min, 2 ml of each of the activated dextran was added to 2 ml of 0.5 M ADH. Each solution was left overnight at 4° C. and then extensively dialyzed to remove free ADH. The hydrazide to dextran ratio was determined using the TNBS assay for hydrazides and a resorcinol/sulfuric acid assay for dextran. The results are summarized in Table 1. There difference between the activation levels achieved with each CDAP was within experimental error. The level of derivatization was essentially the same, indicating comparable levels of polysaccharide activation. Results are depicted in Table 1.

TABLE I

| CDAP Source | ADH/Dextran |
|---|---|
| CDAP— BR | 696 |
| CDAP—TFB | 737 |

### Example 4 Conjugation of BSA to Activated Dextran

This is an indication that there is no difference in the ability of CDAP—TFB and CDAP—Br to activate polysaccharides.

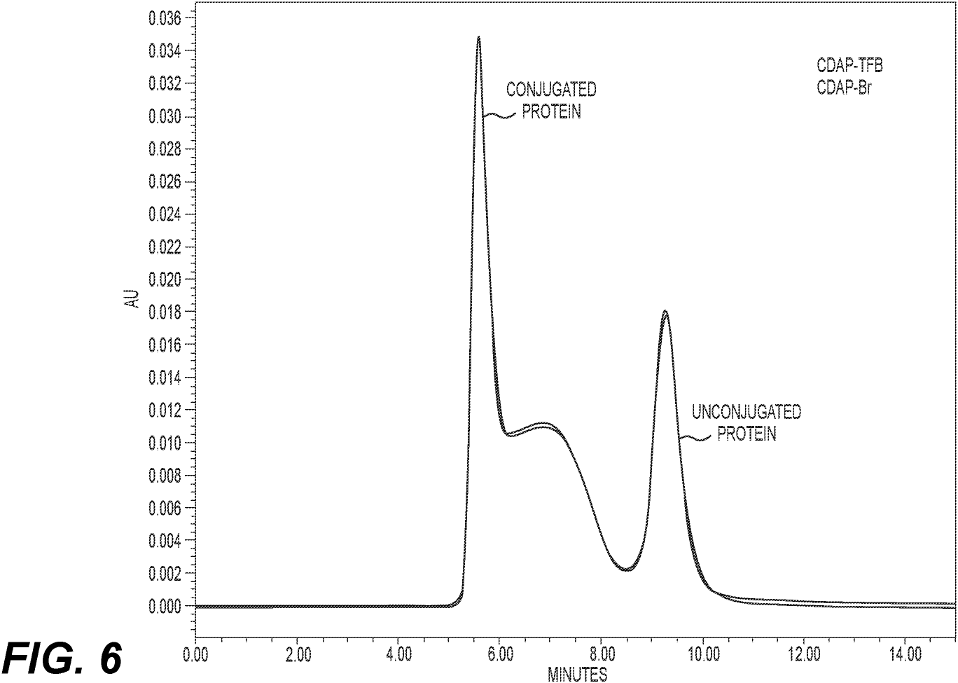

The dextran activated with either CDAP—TFB or CDAP—Br prepared in Example 3 was used for conjugation to TNP₁—BSA. TNP₁—BSA is BSA which has been derivatized with an average of 1 trinitrophenol (TNP) group per BSA molecule. The absorbance of the TNP at 420 nm is therefore a proxy for the BSA. After 15 min of activation, 1 ml of the activated dextran was added to 1 ml of 10 mg/ml TNP₁—BSA in saline. The final pH was about 8.8. The solution was kept at 4° C. overnight. The TNP₁-BSA-Dex conjugate made with each activating reagent was analyzed by SEC HPLC (Tosoh G4000SWXL) at 420 nm where the TNP absorbs but residual DMAP does not. DMAP absorbs at 280 nm, the same wavelength as protein. With the standard protein monitoring at 280 nm, DMAP co-eluting with protein cannot be distinguished from absorbance due to the protein. As shown by the overlapping peaks shown in the SEC HPLC chromatogram of FIG. 6, conjugation produced using CDAP—TFB was virtually identical to conjugation produced using CDAP—Br.

### Example 5. Comparison of CDAP-TFB With CDAP-Br Activation and Conjugation.

CDAP-TFB and CDAP-Br were used to activate a variety of bacterial polysaccharides that are used in typical vaccines. A number of different proteins were conjugated to the activated polysaccharides. Meningococcal serotype A (MenA) is a sialic acid, pneumococcal polysaccharide serotype 22F is a carboxylated polysaccharide, while Hib PRP is a polyribosyl-ribitol-phosphate. MenA and Hib PRP are pH sensitive polymers. As carrier proteins, we used ECOCRM® (CRM₁₉₇) and tetanus toxin heavy chain fragment C (TTHc). The TTHc was derivatized with adipic dihydrazide so that the protein was coupled to the activated polysaccharide via a spacer using a protocol similar to Micoli et al. Vaccine 29 (2011) 712-720 (incorporated by reference). The polysaccharide solutions were prepared with a DMAP buffer (Lees et al. Vaccines 2020, 8, 777; doi:10.3390/vaccines8040777; incorporated by reference) and the activation process performed at 0° C. Each of the three polysaccharides was activated at pH 8.5 with either CDAP—TFB at 100 mg/ml in acetonitrile or CDAP—Br at 200 mg/ml in 0.1 M HCl. To account for the addition of 0.1 M HCl with the CDAP—Br, an equal volume of 0.1 M NaOH was added immediately following the addition of the CDAP—Br. By preparing the CDAP—Br at twice the concentration as CDAP—TFB, the volumes added were identical for the two reagents. This method allowed for the facile substitution of CDAP—Br for CDAP—TFB in the process.

MenA polysaccharide was prepared in water (5 mg/mL, 1 mL) and 150 µL DMAP solution (1.8 M in 0.5xPBS, pH 8.4) was added. The resulting solution was then chilled to 4° C. A solution of CDAP—Br in 0.1 N HCl (200 mg/mL, 50 µL) was then added and followed by addition of 50 µL 0.1 N NaOH. The pH of the reaction was maintained at pH 8.5 for 15 min by slow addition of 0.1 N NaOH at 4° C. After 15 min, a solution of TTHc—ADH (3.8 mg/mL, 1 mL) containing 10% glycerol was added, followed by the addition of 400 µL 5xPBS (pH 8.6) containing 10 mM EDTA. After 3 h at room temperature, the reaction was quenched with 1 M glycine in PBS (2 mL, pH 8.6) overnight at 4° C. The resulting conjugates were dialyzed in 10 kDa cutoff dialysis membrane. Aliquots of samples were analyzed by SEC-MALS analysis on a TOSOH G4000PWxL column with PBS containing 0.02% NaN₃ as the running buffer at 0.5 mL/min. A conjugate analysis was performed upon the conjugate signal in the SEC-MALS spectra. For the CDAP—TFB conjugation, experiment was performed similarly except that CDAP was dissolved in acetonitrile at 100 mg/mL and 100 µL was added to the MenA solution with no NaOH solution added.

Activation and conjugation of Pn22F was carried out similarly to the procedures described for the conjugation of TTHc—ADH to MenA except that two 1 ml tubes of the Pn22F polysaccharide were prepared at 2.1 mg/mL. DMAP was added to each tube, followed by 40 µL CDAP—Br (200 mg/mL in 0.1 M HCl) plus 40 µL 0.1 N NaOH or 80 µL CDAP—TFB (100 mg/mL in acetonitrile). After 15 min with the pH maintained at 8.5, 2.2 mg ECOCRM® was added for each conjugation.

Hib PRP activation and conjugation were carried out similarly to the procedures described for the conjugation of TTHc—ADH to MenA. The Hib PRP polysaccharide was prepared as two 1 ml tubes at 1.5 mg/mL. Activation and conjugation were carried out as described for Pn22F. SEC HPLC MALS analysis of conjugates with polysaccharide activation with CDAP—TFB or CDAP—Br. The results of these experiments are summarized in Tables 2 and 3.

TABLE 2

Activation with CDAP—TFB

| Carrier PS | PS | PS:Protein mg/mg | MW kDa | Polydispersity |
|---|---|---|---|---|
| TTHc—ADH | MenA | 1.3 | 1,500 | 1.771 |
| ECOCRM® | Pn22F | 1.4 | 1,410 | 1.460 |
| ECOCRM® | Hib/PRP | 1.1 | 964 | 1.640 |

TABLE 3

Activation with CDAP—Br

| Carrier PS | PS | PS:Protein mg/mg | MW kDa | Polydispersity |
|---|---|---|---|---|
| TTHc—ADH | MenA | 1.2 | 1,800 | 1.809 |
| ECOCRM® | Pn22F | 1.0 | 1,560 | 1.494 |
| ECOCRM® | Hib/PRP | 1.4 | 942 | 1.623 |

The protein:polysaccharide ratios and conjugate molecular weights were comparable. The polydispersity of conjugates made with CDAP—TFB and CDAP—Br were similar.

### Example 6 Conjugation Methods and Derivatization of Polysaccharide With a Functional Group

Pneumococcal PS serotype 14 is solubilized in water at 5 mg/ml. 3 ml of solution is kept on ice and made 0.2 M DMAP, pH 9. At time 0, 75 µl of CDAP—Br (200 mg/ml in 0.1 M HCl) is added, followed by 75 µl of 0.1 M NaOH. The pH is maintained at pH 9. After 15 min, the activated polysaccharide is distributed into tubes (1 ml each), on ice. 1 ml of DBCO-(PEG)₄-amine (an amine click reagent available from Broadpharm; San Diego, CA) is added to tube 1, 1 ml of biotin-hydrazide (available from Thermofisher; Waltham, MA) is added to tube 2, and 1 ml of fluorescein-hydrazide (available from Thermofisher) is added to tube 3. Each reagent is at about 25 mM final concentration. Each of the reaction mixtures is maintained at pH 9. After 4 hrs, each is extensively dialyzed to remove unreacted components.

DBCO derivatized polysaccharide is used to conjugate to an azide-derivatized protein. Examples of conjugating click reagent derivatized proteins and polysaccharides are described in U.S. Pat. Application Publication No. US2018/03484 (incorporated by reference). Biotin-derivatized polysaccharide are complexed with streptavidin and streptavidin-like proteins such as rhizavidin, as described in U.S. Pat. No. 10,766,932 (incorporated by reference). Fluorescent polysaccharides are used in diagnostic assays.

### Example 7. Amino Derivatization of a Polysaccharide

Pneumococcal serotype 14 is activated with CDAP—Br as in Example 6. After 15 min of activation, an equal volume of 0.5 M hexanediamine is added, and the pH is maintained at 9 for 6 hrs. The reaction mixture is then extensively dialyzed to remove reactants. The product is a polysaccharide derivatized with primary amines. These primary remains are reacted with NHS esters of biotin, fluorescent molecules, click reagents, and/or other crosslinking reagents.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. A process of aqueous conjugation comprising:
reacting 1-cyano-4-dimethylaminopyridinium bromide with a polysaccharide to form an activated polysaccharide;
reacting the activated polysaccharide with a first chemical compound; and
forming a conjugate.

2. The process of claim 1, wherein the polysaccharide comprises a natural or synthetic carbohydrate, oligosaccharide, or a dextran, or a combination thereof.

3. The process of claim 1, wherein the polysaccharide contains an epitope of a pathogen.

4. The process of claim 3, wherein the pathogen comprises Staphylococcus spp., Methicillin-resistant Staphylococcus aureus, Haemophilus spp., Haemophilus influenzae type B, Klebsiella spp., Pseudomonas spp, Escherichia spp., Escherichia coli, Neisseria spp., Neisseria meningitides, Streptococcus spp., Streptococcus pneumococcus, Group B Streptococcus, Mycobacterium spp., Mycobacterium tuberculosis, Acinetobacter spp., Acinetobacter baumannii, Clostridium spp., Clostridium difficile, Burkholderia spp., Burkholderia cepacia, Vibrio spp., Salmonella spp., or Shigella spp.

5. The process of claim 1, wherein polysaccharide or the activated polysaccharide contains a second chemical compound.

6. The process of claim 5, wherein the second chemical compound comprises a linker molecule.

7. The process of claim 1, wherein the linker molecule comprises a homobifunctional or a heterobifunctional linker molecule.

8. The process of claim 1, wherein the first chemical compound comprises a natural or recombinantly or synthetically produced peptide, polypeptide, or protein.

9. The process of claim 1, wherein the first chemical compound comprises a natural or recombinantly produced diphtheria toxoid, diphtheria cross reactive material (CRM), CRM₁₉₇. tetanus toxoid, tetanus toxin heavy chain protein, Pseudomonas exoprotein A, Pseudomonas aeruginosa toxoid, Bordetella pertussis toxoid, or Clostridium perfringens toxoid.

10. The process of claim 1, wherein the 1-cyano-4-dimethylaminopyridinium bromide contains an approximate ratio of about one mole of 1-cyano-4-dimethylaminopyridinium per mole of bromide anion.

11. The process of claim 1, which does not include tetrafluoroborate or an organic solvent.

12. The process of claim 1, further comprising removing components with a lower molecular weight than the conjugate by dialysis, filtration, chromatography, or a combination thereof.

13. A conjugate produced by the process of claim 1.

14. The conjugate of claim 13, which is an antigenic compound, an immunological compound, a diagnostic agent, or a vaccine.

15. A process of conjugation under aqueous conditions comprising:
reacting 1-cyano-4-dimethylaminopyridinium bromide with a polysaccharide containing biotin or a biotin-like compound to form activated biotinylated polysaccharide;
reacting the activated polysaccharide with a biotin hydrazide; and
reacting the activated polysaccharide with a first chemical compound forming a biotinylated polysaccharide conjugate.

16. The process of claim 15, wherein the polysaccharide comprises a natural or synthetic carbohydrate, oligosaccharide, or dextran, or a combination thereof.

17. The process of claim 15, wherein the first chemical compound comprises a natural or recombinantly or synthetically produced peptide, polypeptide, or protein.

18. The process of claim 15, wherein the first chemical compound comprises a natural or recombinantly produced diphtheria toxoid, diphtheria cross reactive material (CRM), CRM₁₉₇. tetanus toxoid, tetanus toxin heavy chain protein, Pseudomonas exoprotein A, Pseudomonas aeruginosa toxoid, Bordetella pertussis toxoid, or Clostridium perfringens toxoid.

19. The process of claim 15, wherein the biotinylated polysaccharide conjugate is coupled to an affinity compound.

20. The process of claim 19, wherein the affinity compound comprises an avidin, an avidin-like protein, a rhizavidin, a streptavidin, or a functional portion thereof.

21. The process of claim 15, further comprising removing components with a lower molecular weight than the conjugate by dialysis, filtration, chromatography, or a combination thereof.

22. A conjugate produced by the process of claim 15.

23. The conjugate of claim 22, which is an antigenic compound, an immunological compound, a diagnostic agent, or a vaccine.

24. A process of conjugation under aqueous conditions comprising:
reacting 1-cyano-4-dimethylaminopyridinium bromide with a polysaccharide to form a cyanalyated polysaccharide;
reacting the cyanalyated polysaccharide with a dibenzocyclootyne compound to a form a dibenzocyclootyne coupled cyanalyated polysaccharide; and
reacting the dibenzocyclootyne coupled cyanalyated polysaccharide with a first chemical compound protein to form a conjugate.

25. The process of claim 24, wherein the polysaccharide comprises a natural or synthetic carbohydrate, oligosaccharide, or a dextran, or a combination thereof.

26. The process of claim 24, wherein the dibenzocyclootyne coupled cyanalyated polysaccharide further comprises polyethylene glycol.

27. The process of claim 24, wherein the first chemical compound comprises a natural or recombinantly produced diphtheria toxoid, diphtheria cross reactive material (CRM), CRM₁₉₇. tetanus toxoid, tetanus toxin heavy chain protein, Pseudomonas exoprotein A, Pseudomonas aeruginosa toxoid, Bordetella pertussis toxoid, or Clostridium perfringens toxoid.

28. A conjugate produced by the process of claim 24.

29. The conjugate of claim 27, which is an antigenic compound, an immunological compound, a diagnostic agent, or a vaccine.

30. An aqueous composition comprising a polysaccharide activated with 1-cyano-4-dimethylaminopyridinium in the presence of bromide anions.
